(19) United States
(12) Patent Application Publication
(10) Pub. No.: US 2005/0203379 A1
(43) Pub. Date: Sep. 15, 2005

(54) Method and apparatus for MR image evaluation by mapping perfusion-weighted and diffusion-weighted MR images

(76) Inventor: Gudrun Graf, Erlangen (DE)

Correspondence Address: SCHIFF HARDIN LLP;Patent Department, 6600 Sears Tower, 233 South Wacker Drive, Chicago, IL, 60606, US

(21) Appl. No.: 11/049,087

(22) Filed: Feb. 2, 2005

(30) Foreign Application Priority Data
Feb. 2, 2004 (DE) 10 2004 005 122.4

(51) Int. Cl. A61B 5/05

(52) U.S. Cl. 600/411

(57) ABSTRACT

In a method and apparatus for improved evaluation of magnetic resonance images, in particular for detection and delineation of endangered brain tissue, a perfusion-weighted MR image and a diffusion-weighted MR image of the same body region are superimposed to form a mismatch image.

### BACKGROUND OF THE INVENTION

1. Field of the Invention

The present invention concerns a method for improved evaluation of magnetic resonance images, in particular for detection and delineation of endangered (at-risk) brain tissue, as well as an apparatus for implementation of this method.

2. Description of the Prior Art

In order to be able to better represent tissue, in particular brain tissue, with the details of interest for a physician, various MR imaging methods are known with which particular tissue areas can be represented preferentially with different weighting of their respective states.

In MR perfusion imaging, tissue known as the penumbra or the “tissue-at-risk” is shown. With the help of an MR diffusion-weighted exposure, an irreversibly damaged area can be shown very well. In order to evaluate these different exposures a visual comparison of the images must be undertaken, which is a significant problem in practice.

### SUMMARY OF THE INVENTION

An object of the present invention is to provide a method and an apparatus that allow improved evaluation of magnetic resonance images, in particular for detection and delineation of endangered brain tissue, that makes superfluous the laborious and always-imprecise visual comparison in detail of different images with one another.

This object is achieved in accordance with the invention in an apparatus wherein a perfusion-weighted MR image and a diffusion-weighted MR image of the same body region are superimposed to form a mismatch image, with the structures in both MR images being preferably given different colors before the mixing.

By the superimposition of the images, the observer immediately recognizes which area (for example given a stroke) could be saved, i.e., an area that is not irreversibly damaged.

Details of techniques and apparatuses for conducting MR perfusion studies are described, for example, in U.S. Pat. Nos. 6,295,465 and 6,556,855 and 6,828,788. Details of techniques and apparatuses for generating MR diffusion-weighted images are described, for example, in U.S. Pat. Nos. 6,265,872 and 5,492,123. These teachings of each of these five patents are incorporated herein by reference.

The inventive method includes the steps of generating a mismatch map by superimposed mapping of a perfusion-weighted MR image and a diffusion-weighted MR image of the same body region, preferably contrasted in color, on an image medium (image carrier).

### DESCRIPTION OF THE DRAWINGS

FIG. 1 shows a magnetic resonance (MR) image of a brain region reconstructed from a diffusion examination (scan).

FIG. 2 shows a map of an MR image of the same brain region reconstructed from a perfusion examination (scan).

FIG. 3 shows a mismatch map, i.e. a representation of the images according to FIGS. 1 and 2 superimposed on an image medium in accordance with the invention.

### DESCRIPTION OF THE PREFERRED EMBODIMENTS

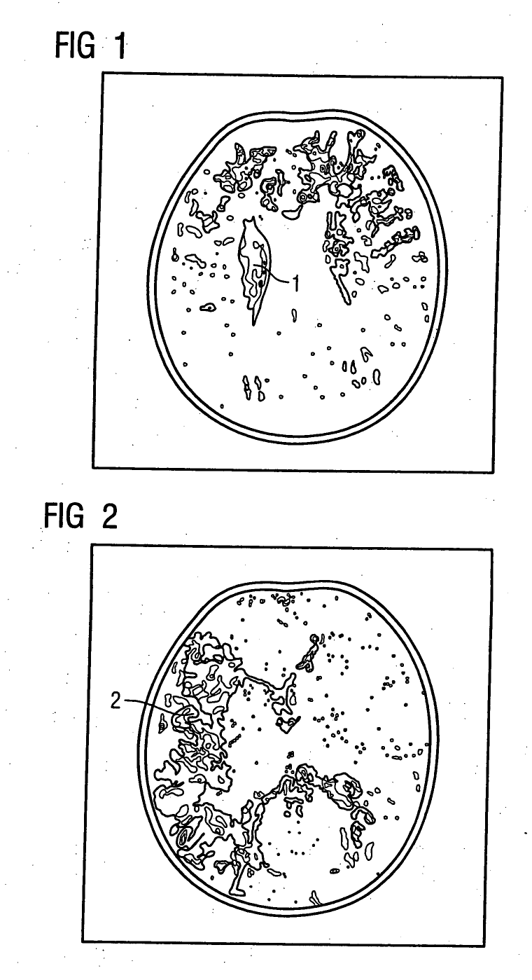

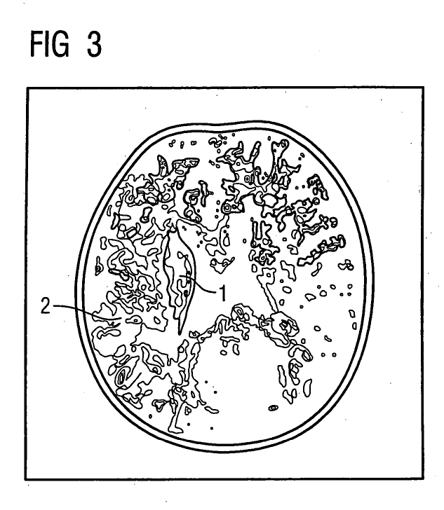

The dark area in the diffusion image according to FIG. 1 shows the brain area that most likely can no longer be rescued (already necrotized) for the patient. The area detectable in the perfusion image according to FIG. 2, within which the dead area 1 is located, will also not be recoverable if the patient is not treated. By the superimposition of the perfusion image according to FIG. 2 with the diffusion-weighted image according to FIG. 1, the physician at a glance can recognize the position of the damaged brain area and thereby also far better assess where the critical points are and what tissue can still actually be saved.

In practice, the dark, dead tissue area 1 and the lighter, recognizably more lightly damaged tissue area 2 are displayed with contrasting colors, achieving evaluation possibilities that are even better and are in more detail when the color intensity or the color gradation is modified as well depending on the shading degree.

Although modifications and changes may be suggested by those skilled in the art, it is the intention of the inventor to embody within the patent warranted hereon all changes and modifications as reasonably and properly come within the scope of her contribution to the art.

1. A method for evaluating a magnetic resonance image comprising the steps of:
obtaining a perfusion-weighted MR image of a body region of a subject;
obtaining a diffusion-weighted MR image of said body region; and
superimposing said perfusion-weighted MR image and said diffusion-weighted MR image to form a mismatch image for evaluation thereof.

2. A method as claimed in claim 1 comprising obtaining said perfusion-weighted MR image and said diffusion-weighted MR image of a body region containing brain tissue of said subject, and evaluating said mismatch image to identify endangered brain tissue.

3. A method as claimed in claim 1 comprising selectively coloring structures in said perfusion-weighted MR image with a first color and selectively coloring selected structures in said diffusion-weighted MR image with a second color, before superimposing said perfusion-weighted MR image and said diffusion-weighted MR image.

4. An apparatus for evaluating magnetic resonance images comprising:
a processor supplied with a perfusion-weighted MR image of a body region of an examination subject and with a diffusion-weighted MR image of said body region, said processor superimposing said perfusion-weighted MR image and said diffusion-weighted MR image to form a mismatch image; and
a display connected to said processor at which said mismatch image is displayed for evaluation thereof.

5. An apparatus as claimed in claim 4 wherein said processor colors selected structures in said perfusion-weighted MR image with a first color and colors selected structures in said diffusion-weighted MR image with a second color before superimposing said perfusion-weighted MR image and said diffusion-weighted MR image.
